(19) United States
(12) Patent Application Publication
(10) Pub. No.: US 2009/0185998 A1
(43) Pub. Date: Jul. 23, 2009

(54) NEW ACTIVATED POLY(ETHYLENE GLYCOLS) AND RELATED POLYMERS AND THEIR APPLICATIONS

(75) Inventors: Francesco Maria Veronese, Padova (IT); Gianfranco Pasut, Fiume Vento (IT); Giancarlo Tonon, Pula (IT); Rodolfo Schrepfer, Villa Guardia (IT)

Correspondence Address: DARBY & DARBY P.C., P.O. BOX 770, Church Street Station, New York, NY, 10008-0770, US

(73) Assignee: Bio-Ker S.r.I, Gessate (IT)

(21) Appl. No.: 12/295,450

(22) PCT Filed: Dec. 29, 2006
(86) PCT No.: PCT/EP2006/070265

§ 371 Date: Nov. 12, 2008

(30) Foreign Application Priority Data
Mar. 30, 2006 (IT) MI2006A000612

(51) Int. Cl. A61K 38/21; C07K 2/00; C07H 21/00; C07K 14/00; C07K 14/62; C12N 9/96; C07K 14/52; A61K 47/10; C08G 65/333

(52) U.S. Cl. 424/85.7; 530/300; 536/22.1; 530/385; 530/303; 435/188; 530/351; 514/788; 525/408

(57) ABSTRACT

There are disclosed chemically active poly(ethylene glycols) and other hydrophilic polymers that are suitable for coupling to pharmaceutically or diagnostically active agents such as peptides, oligonucleotides, proteins or non-peptide molecules. The compounds are represented by the formula Poly-(X—NH—CO-A)ₙ wherein, Poly is a hydrophilic polymer having a molecular weight of from about 300 to 100000 Daltons; A together with —NH—CO— forms a reactive group; X is a spacer moiety or a bond; n is an integer comprised between 1 and 50. The active agents of interest which may be conjugated to the disclosed compounds may be selected from hemoglobin, insulin, urokinase, alpha-interferon, G-CSF, hGH, asparaginase, adenosine deaminase, superoxide dismutase and catalase.

This invention relates to preparation of novel activated poly(ethylene glycol) and other hydrophilic polymers and the use of them to modify a biomaterial.

### BACKGROUND OF THE INVENTION

It is known that polyethylene glycols (PEGs), also known as poly(ethylene oxide) (PEO), are linear, flexible polymers, available in a great range of molecular weights and largely employed in many pharmaceutical preparations, for example formulations to be administered by parenteral, topic, ophthalmic, oral and rectal route. They correspond to the general formula HOCH₂(CH₂OCH₂)ₘCH₂OH or in methoxylated form CH₃OCH₂(CH₂OCH₂)ₘCH₂OH, wherein m represents the average number of the polyoxyethylene moieties. PEGs are stable and show a good compatibility with tissues and mucosae. According to their molecular weight, they can exist in several forms. In this way, PEGs of from 200 Dalton (Da) to 600 Da are liquids, PEGs with molecular weight higher than 1000 Dalton are solids of wax type, whereas with 6000 Da and more are free-flowable powders.

PEGs have a low toxicity in oral, parenteral and topic applications. After intravenous administration in human beings, PEGs with molecular weight of from 1000 Da to 10000 Da are quickly excreted, predominantly by renal route; those having a higher molecular weight with a decreasing rate while the molecular weight increases.

PEGs are employed in aqueous solutions as agents for adjusting viscosity and respectively their consistency. At concentrations ranging about 30%, they are used also for parenteral solutions. In solid pharmaceutical forms, PEGs with high molecular weight can increase the binder efficiency, thus conferring plasticity to grains. Those with high molecular weight are above all employed also as lubricants (Handbook of excipients 2000, 392-398).

Poly(ethylene glycols) (PEGs) and derivatives thereof are having increasing interest in chemical, biomedical, and other industrial applications due to their useful properties, such as, amphiphilic behavior, solubility in aqueous and organic solvents, high purity, low polydispersivity, biological compatibility. Since they can be activated for conjugation to other compounds, such polymers have been employed for example, as drug carriers, matrices for liquid phase peptide or polynucleotides synthesis, surfaces modifying agents, and to prepare conjugate with peptide and protein (See, e.g., Roberts M J et al., Adv. Drug Del. Rev., 54: 459-476, 2002; Veronese F. M, Biomaterials, 22: 405-417, 2001).

In fact PEG attachment to proteins and peptides can improve, besides their solubility, stability and resistance to proteolytic inactivation, pharmacokinetic properties and moreover for diminishing immunogenicity and antigenicity (Delgado C. et al., Critical Rev Ther Drug Carrier Syst 1992, 9, 249-304; Adv. Drug reviews 2002, 54, 453-606; Harris J M, Chess R B. Effect of PEGylation on Pharmaceuticals. Nature Reviews Drug Discoveries 2003, 2, 214-221. Veronese F. M, Pasut G., Drug Disc Today 2005, 10, 1451-1458).

It has been suggested that the mentioned effects are due to the PEG and to its strictly connected water molecules that cover and protect by a shielding effect the bound molecule, thus preventing proteolytic enzymes approach, immune system cells, receptors and other tissues constituents contact. Furthermore, the molecular weight increase reduces the glomerular filtration with consequent increase of plasmatic half-life and improvement of conjugates pharmacokinetics.

In U.S. Pat. No. 4,179,337 to Davis et al. it is disclosed that proteins linked to PEG possess prolonged in vivo half-life because the reduced kidney clearance and immunogenicity. Moreover, in literature several examples of compounds of proteic nature having interesting biological properties, obtained by genetic engineering also, are described. Among said compounds hemoglobin, insulin, urokinase, alpha-interferon, G-CSF, hGH, asparaginase, adenosine deaminase, superoxide dismutase (metalloenzyme catalysing the dismutation reaction of superoxide radical in hydrogen peroxide and molecular oxygen, later on SOD), catalase, etc, can be mentioned (Pasut G. et al. Expert Op Ther Patents 2004, 14, 859-894). For example the antioxidant enzymes SOD and catalase could be employed in the treatment of rheumatoid arthritis, ligaments degenerative disease, ischemia and vascular injuries in general. However, the therapeutic strength of native proteins is highly restricted by their short half-life and by possible allergic side reactions. These problems can be overcome conjugating said proteins with PEG (by a process most known as PEGylation), and in fact several PEGylated proteins have been approved by FDA, in particular PEG-adenosine deaminase, PEG-interferons, PEG-asparaginase and PEG-G-CSF. Oligonucleotides were PEGylated also and one of such products is already marketed under the trade name of Macugen.

To link PEG to a molecule it is necessary that the polymer has an “active group” at the terminus suitable for a reaction with a group on the recipient molecule. With new discoveries in medical research and development of nanotechnology tools, there is a growing demand for new and improved PEG derivatives with different properties which can be tailored to meet user requirements.

Several are the amino acid residues in a protein that can be PEGylated by chemical procedures or enzymatic ones, but the amines are those that attracted most the researchers mainly because they are commonly present in proteins and exposed to solvents and furthermore because the amino acylation or alkylation reactions are well known in literature (Harris J M, Chess R B. Nature Reviews Drug Discoveries 2003, 2, 214).

For instance Clark R. proposed a PEGylation of human growth hormone with N-hydroxysuccinimide ester of poly(ethylene glycol) 5000 Da (mPEG-CO—NHS) using PEG/protein molar ratio ranging from 10 to 30 (Clark R. J Biol Chem 1996, 271(36), 21969-21977), obtaining a wide mixture of isomers mainly composed of multi-PEGylated conjugates, thus proving that when using a high reactive PEG, as the activated PEG-aliphatic acid (i.e. mPEG-CO—NHS), many polymer chains are attached to a protein making difficult to obtain monoPEGylated species.

WO 90/13540 (1990) to Zalipsky S. discloses the preparation of succimidyl carbonate PEG (SC-PEG) and its conjugation to proteins and polypeptides. The “activated PEG derivative” reacts fast with amino groups of polypeptides and U.S. Pat. No. 5,951,974 (1999) discloses a method to preferentially direct the PEG attachment to histidine (at least 30% of total PEG linked) of the alpha-interferon, a link that possesses the capacity to be hydrolyzed in vivo yielding the starting native protein.

However, the SC-PEG derivatives still reacts too fast with nucleophylic groups in a protein, thus reducing the possibility of the PEG derivative to discriminate among the different reactive amino acid residues in a protein and therefore leading to a complex mixture of PEGylated-protein isomers (Wang Y., et al. Adv Drug Del Rev 2002, 54, 547-570). In the monoPEG-interferon conjugates mixture there is a isomer in which the polymer is linked by a labile bond to the histidine 34 (about 40% of the total amount of isomers), even though the restore of a fully active protein is wanted it can, on the other side, prevent the fully exploitation of the prolonged conjugate half-life in blood reached by stable polymer links. In fact after hydrolysis the native protein undergoes rapid kidney clearance. Therefore, a more stable PEG-protein linkage at the level of histidine, but still able to release the native protein with a slower rate is wanted, together with a higher degree of conjugation to this amino acid thus better joining the two contrasting factors of a prolonged half-life of conjugate and a higher activity of native protein. Recently, several system for protein release from conjugated polymer have been proposed (Greenwald R B, et al. Bioconjugate Chem 2003, 14:395-403; Greenwald R B, et al. J Med Chem 2000, 43:475-487; Tsubery H, et al. J Biol Chem 2004, 279(37):38118-38124), but all of them are involving aromatic spacers that may arise immunogenicity concerns after conjugation to a protein. Moreover, in most of them the protein release is accomplished by enzyme controlled cleavage, and because the enzymes concentrations are different in each human being this may lead to different protein release rate and therefore therapeutical response. U.S. Pat. No. 6,214,966 (2001) to J. M. Harris discloses a PEG and related polymer derivatives useful for conjugation to protein or other pharmaceuticals, forming conjugates from which the bound drug is released by water hydrolysis of an unstable linkage close to the active group of the polymer; the release leaves a small moiety, before belonging to the polymer, linked to the drug, thus arising immunogenetic concerns especially for modification of protein.

Therefore, an aliphatic spacer able to release the conjugated drug, in the native form, under the control of a predetermined and the common hydrolytic cleavage is welcome.

### SUMMARY OF THE INVENTION

The invention provides chemically active poly(ethylene glycols) (PEGs) and related polymers that are suitable for coupling to drugs, including proteins, enzymes, small molecules, and others to give water-soluble conjugates.

The PEG and related polymer derivatives of the invention contain a weak active group, which allows discrimination among all of the available groups in a drug as a protein, where for example an amino modification with the common PEG acylating derivatives usually lead to a wide mixture of isomers. These derivatives provide a sufficient circulation time for the PEG-drug conjugates, and when PEG is linked to an imadozole residue (e.g. of a histidine) the bound drug is released in the surrounding environment by a hydrolytic breakdown with a predetermined rate. Methods of preparing this new active PEGs and related polymers, and methods of preparing the PEG conjugates are also included in the invention.

By conjugation of these activated PEGs and related polymer derivatives to a drug it is possible to impart water solubility, increased size, reduced kidney clearance, stability and reduced immunogenicity to the conjugate. Linking these active derivatives through an imidazole residue or similar molecules or an alcohols it is also possible to provide a controllable hydrolytic release of the bound drug in an aqueous environment by a proper design of the linkage. The activated derivatives of this invention can be used to discriminate among all reactive groups in a drug, because their lower reactivity allows them to react preferentially with the most reactive and/or exposed group of a selected drug, for example but not exclusive these PEG derivatives can mainly link the highest reactive amino groups of a protein. The derivatives of this invention can be used to increase solubility and increased blood half-life of proteins, peptides and non-protein drugs and then eventually control the release of the drugs from the polymer. According to this invention, the drugs that previously had reduced biological activity, when permanently conjugated to a polymer, can now have an enhanced activity if coupled to these PEG and polymer derivatives by the, above mentioned, releasable manner (for example but not exclusive, an imidazole linkage).

In general form, the activated derivatives of the invention can be described by the following equations:

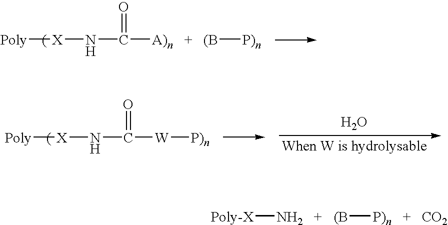

In the above equations:
“Poly” is an hydrophilic polymer having a molecular weight of from about 300 to 100000 Daltons;
“A” together with —NH—CO— forms a reactive group;
“X” is a spacer moiety or a bond;
“n” is an integer comprised between 1 and 50, preferably between 1 and 10, even more preferably between 1 and 5, and even more preferably 1, which represents the number of chemically active end groups present on Poly.

(B—P)ₙ is a molecule for a conjugation to Poly, in which P is an active drug and B is a reactive group of the same drug that is reactive with A and that can be naturally included in P or intentionally linked to it, including, for example, a protein P in which B is an imidazole residue of a histidine or an amine group.

“W” represents the new linkage formed by reaction of A and B, which can be reasonable stable in water, when B is an amino group, or hydrolysable in water when B is the secondary amine of the imidazole residue of histidine or a molecule having a similar structure or an alcohols.

Examples of P are proteins, peptides, oligonucleotides, and other pharmaceuticals. A may be for instance group reactive toward B, in some examples A is N-hydroxysuccinimide, N-hydroxybenzotriazole or p-nitrophenol while B is represented by amines or alcohols. Examples of W include urea formed by reaction of active carbamates with amines or urethanes from reaction between active carbamates and hydroxyl groups. W can be hydrolysable in water, for example the urea formed by reaction of active carbamates with the amine of the imidazole residue of a histidine (scheme A) or reasonable stable in the case of amino groups (scheme B). In any case the linking of these new PEG and polymer derivatives is limited or preferentially direct to the most reactive and exposed group in the selected drug, thanks to the lower reactivity of the polymers objects of this invention.

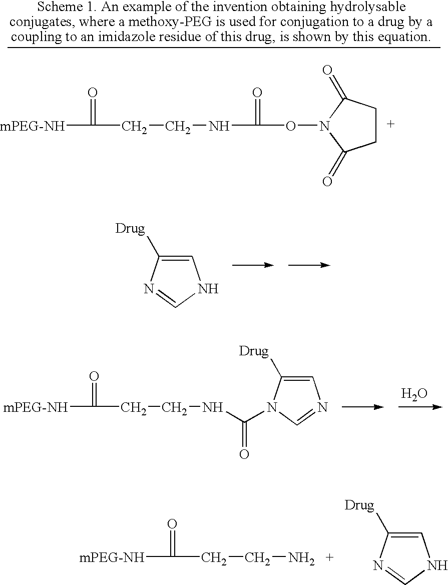

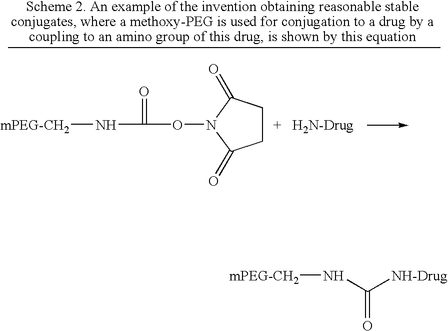

The protein is released by hydrolytic breakdown in its native form, without any additional molecule attached to it.

The invention provides activated PEGs and related polymers in which a weak reactive carbamate allows a better discrimination among all of the available different groups in a multifunctional drug, such as a protein, and when the site of linking of the drug is the amine of an imidazole (i.e. histidine) the obtained conjugate can release the native drug following hydrolysis. Furthermore, the rate of the hydrolysis can be tailored by a suitable chemical moiety close to the active group of the polymer.

The foregoing and other objects, advantages, features of the invention and the manner in which the same are accomplished will be more explained in the following detailed description of the invention.

### DETAILED DESCRIPTION

The object of the present invention is represented by compounds of formula

Poly-(X—NH—CO-A)ₙ

wherein:

Poly is an hydrophilic polymer having a molecular weight of from about 300 to 100000 Daltons;

A together with —NH—CO— forms a reactive group, and in preferred embodiments A is selected among N-hydroxysuccinimide, N-hydroxybenzotriazole or p-nitrophenol.

X is a spacer moiety or a bond;

n is an integer comprised between 1 and 50, preferably between 1 and 10, more preferably between 1 and 5, and even more preferably it is equal to 1, which represents the number of chemically active end groups present on Poly.

According an embodiment of the invention, X is selected from:

### a) —NH—CO—CH(R1)-CH(R2)

wherein R1 and R2, independently from each other, are selected from: H, an optionally substituted alkyl group, an optionally substituted aryl group, an optionally substituted aryl-alkyl group, an hydroxy group, an amino group and/or a carboxy group;
b) an alkyl group optionally substituted by one or more groups preferably selected from hydroxy, an amino or carboxy groups; or
c) an aryl group.

According to other embodiments, X may be a C₂-C₁₀ alkyl group, R1 and/or R2 may be H or a C₂-C₁₀ alkyl group. X can also be a molecule as a peptide or an oligonucleotides. Poly may be a linear or branched poly(ethylene glycol) or a derivative thereof, preferably selected from methoxy-poly(ethylene glycol) or diol-poly(ethylene glycol); in particular, the poly(ethylene glycol) may have a molecular weight of from about 10000 to 60000 Daltons, preferably from 5000 to 40000 Daltons.

Another embodiment is represented by a method for the preparation of a conjugate between a pharmaceutically or diagnostically active agent and a compound according to the present invention, said method comprising:

mixing the pharmaceutically or diagnostically active agent and the compound according to the invention;

isolating the final conjugate.

Preferably, the mixing is carried out in water or buffer solutions, at a temperature of between 3-40° C., for a period of 1-3 hours; on its turn, the isolation is preferably performed by precipitation or by chromatographic techniques, such as ionic exchange, gel-filtration or reverse phase chromatographies.

The following detailed description describes several examples of the derivatives disclosed in this invention as represented by the following general equation presented in the summary:

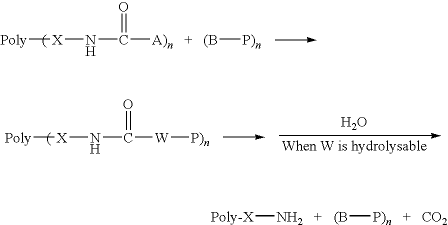

In the following discussion, Poly will often be referred to for convenience as PEG. However, other hydrophilic polymers of similar properties are also suitable for use in the practice of the invention and that the use of the term PEG is intended to be inclusive and not exclusive in this respect.

Polyethylene glycols (PEGs), also known as poly(ethylene oxide) (PEO), are useful in the practice of the invention. PEG is largely employed in many pharmaceutical preparations, for example formulations to be administered by parenteral, topic, ophthalmic, oral and rectal route. PEGs are stable and show a good compatibility with tissues and mucosae. PEGs typically is clear, colorless, odorless, soluble in water, stable to heat, inert to many chemical agents, does not hydrolyze or deteriorate, and is non-toxic. Poly(ethylene glycols) and derivatives thereof are having increase interest in chemical, biomedical, and other industrial applications due to their useful properties, such as, amphiphilic behavior, solubility in aqueous and organic solvents, high purity, low polydispersivity, biological compatibility and since they can be activated for conjugation to other compounds such polymers have been employed for example, as drug carriers, matrices for liquid phase peptide or polynucleotides synthesis, surfaces modifying agents, and to prepare conjugate with peptide and protein. When attached to a moiety having some desirable function in the body, PEG increases the size of the drug, reducing the kidney clearance, tends to mask the drug surface and can reduce or eliminate any immune response so the organism can tolerate the presence of the drug that can thus explicate longer its function thanks to the lower clearance. Accordingly, the activated PEGs of the invention should be substantially non-toxic and should not tend to produce an immune response or other undesirable effects.

Other water soluble polymers than PEG are suitable for similar application, for example poly(propylene glycol) (PPG), poly(vinyl alcohol) (PVA), poly(oxyethylated sorbitol) and the like, poly(oxazoline), poly(acryloylmorpholine) (PAcM), poly(vinylpirrolidone) (PVP). The polymers can be homopolymers or random or block copolymers, with linear or branched structure, or substituted or unsubstituted similar to mPEG and other capped, monofunctional PEGs having a single active site available for attachment to a linker.

By the term “drug” it is intended any substance useful for the diagnosis, treatment, mitigation, cure, or prevention of disease in human and other animals, or otherwise enhance physical or mental well being.

The terms “group”, “functional group”, “moiety”, “active group”, “reactive group” and “reactive site” are all somewhat synonymous in the chemical arts and are used in the art and herein to refer to distinct, definable portions or units of a molecule and to units that perform some function or activity and are reactive with other molecules or portions of molecules.

The term “linkage” is used to refer to groups that normally are formed as the result of a chemical reaction and typically are covalent linkages. Hydrolytically unstable linkages are those that react with water typically causing a molecule to separate into two or more components.

When Poly is PEG, the polymer has preferentially an average molecular weight of at least 1000 Da, preferably of at least 4000, more preferably at least 10000, and even more preferably of at least 20000. In preferred embodiments, Poly is a poly(ethylene glycol) (PEG) having an average molecular weight ranging from 1000 to 40000 Da. Some preferred Poly are PEG10000, PEG20000, PEG30000, PEG40000 either with linear or branched structure.

The invention includes PEGs, as above reported, containing a moiety that predetermines the reactivity of the reactive groups useful for coupling to amines of molecules to be delivered in vivo or into a substance taken from a living entity:

PEG-X—NH—CO-A

Where “X” is a spacer moiety or a bond;

X can also be selected among:
a) —NH—CO—CH(R1)-CH(R2)-, wherein R1 and R2 are independently an H or optionally substituted alkyl, or optionally substituted aryl or optionally substituted aryl-alkyl groups or groups preferably selected among an oxy-, a hydroxyl-, an amino- or a carboxyl-group, when R1=R2=H the β amino acid spacer is β alanine,
b) or X is an alkyl group preferably comprising 2 to 10 carbons optionally substituted by one or more groups preferably selected among an oxy, a hydroxyl, an amino or a carboxyl group,
c) or X is an aryl group.
X can also be a drug, as a peptide or an oligonucleotide; and when the linkage between the PEG derivatives and the bound molecules is hydrolysable in water this can be exploited to predetermine the trigger of the activity of the drug.
Where “A” together with the moiety —NH—CO— forms a reactive group and is preferably selected among the group of N-hydroxysuccinimide, N-hydroxybenzotriazole or p-nitrophenol.

Among preferred embodiments the PEG derivatives have the following formula:

PEG-NH—CO—CH₂CH₂—NH—CO—NHS

PEG-OCH₂CH₂—NH—CO—NHS

PEG-NH—CO—CH(R1)-CH(R2)-N(R3)-CO—NHS

Where R1, R2, and R3 are independently an H or optionally substituted alkyl, or optionally substituted aryl or optionally substituted aryl-alkyl groups or groups preferably selected among an oxy-, a hydroxyl-, an amino- or a carboxyl-group; when R1=R2=R3=H the β amino acid spacer is β alanine.

The invention further explains, with some specific examples of PEG derivatives, their synthesis and the application.

### Example 1

### Preparation of CH₃O—PEG-(CH₂)ₙ—NH—CO—NHS (n=1-4)

Reaction

CH₃O—PEG-(CH₂)ₙ—NH₂+NHS—CO—NHS+pyridine→CH₃O—PEG-(CH₂)ₙ—NH—CO—NHS

CH₃O—PEG-(CH₂)ₙ—NH₂ 20000 (5.0 g, 0.25 mmole, n=1-4) was azeotropically dried with 50 ml of acetonitrile and then slowly cooled to room temperature. To the resulting solution were added disuccinimidyl carbonate (265 mg, 1 mmole) and pyridine (0.25 ml), and the reaction was let to proceed overnight at room temperature. The solvent was then removed under vacuum and 40 ml of dry CH₂Cl₂ were added to the residue. The insoluble solid was removed by filtration and the filtrate was washed with pH 4.5 sodium chloride saturated acetate buffer. The organic phase was dried over anhydrous sodium sulfate, and the solution was concentrated till 15 ml under vacuum. The concentrated solution was dropped over 300 ml of diethyl ether under vigorously stirring. The precipitate was collected by filtration and dried under vacuum. Yield: 4.6 g (92%). ¹H-NMR of CH₃O—PEG-(CH₂)₂—NH—CO—NHS (CDCl₃): δ 3.62 (bs, —O—CH₂—PEG), δ 3.54 (t, —CH₂—CH₂—NH—CO—NHS), δ 2.8 (s, —NHS).

### Example 2

### Preparation of CH₃O—PEG-NH—CO—(CH₂)₂—NH—CO—NHS Via (β-Alanine)

Reactions

CH₃O—PEG-NH₂+NHS—CO—(CH₂)₂—NH—BOC→CH₃O—PEG-NH—CO—(CH₂)₂—NH—BOC

TFA

CH₃O—PEG-NH—CO—(CH₂)₂—NH—BOC→CH₃O—PEG-NH—CO—(CH₂)₂—NH₂

CH₃O—PEG-NH—CO—(CH₂)₂—NH₂+NHS—CO—NHS+pyridine→CH₃O—PEG-NH—CO—(CH₂)₂—NH—CO—NHS

CH₃O-PEG-NH₂ 20000 (5.0 g, 0.25 mmole) was azeotropically dried with 50 ml of toluene and then slowly cooled to room temperature. To the resulting 12 ml solution were added 10 ml of dry CH₂Cl₂, N-hydroxysuccinimide ester of N—BOC-β-alanine (144.7 mg, 0.5 mmole) and Et₃N (70 μl, 0.5 mmole), and the reaction was let to proceed overnight at room temperature. The solution was then filtered and dropped over 300 ml of diethyl ether under vigorously stirring. The precipitate, collected by filtration and dried under vacuum, was then dissolved in 20 ml of a solution of CH₂Cl₂/TFA/H₂O (54.5:45.4:0.1) and stirred for 3 hour at room temperature. The solvent was removed under vacuum and to the obtained oil 20 ml of CH₂Cl₂ was added. To the resulting solution were added disuccinimidyl carbonate (265 mg, 1 mmole) and pyridine (0.25 ml), and the reaction was let to proceed overnight at room temperature. The solvent was then removed under vacuum and 40 ml of dry CH₂Cl₂ were added to the residue. The insoluble solid was removed by filtration and the filtrate was washed with pH 4.5 sodium chloride saturated acetate buffer. The organic phase was dried over anhydrous sodium sulfate, and the solution was concentrated till 15 ml under vacuum. The concentrated solution was dropped over 300 ml of diethyl ether under vigorously stirring. The precipitate was collected by filtration and dried under vacuum. Yield: 4.2 g (84%). ¹H-NMR (CDCl₃): δ 3.62 (bs, —O—CH₂—PEG), δ 3.54 (t, —CH₂—CH₉—NH—CO—NHS), δ 2.5 (t, —NH—CO—CH₂—CH₂—NH—CO—NHS), δ 2.8 (s, —NHS).

### Example 3

### Preparation of CH₃O—PEG-NH—CO—(CH₂)₂—NH—CO—NHS (Via DCC/NHS)

Reaction

CH₃O—PEG-NH₂+NHS+DCC→CH₃O—PEG-NH—CO—(CH₂)₂—NH—CO—NHS

Dicyclohexylcarbodiimide (232 mg, 1.125 mmole) and N-hydroxysuccinimide (130 mg, 1.125 mmole) was dissolved in 15 ml of CH₂Cl₂ and stirred at room temperature for 1 hour. To the solution was then added CH₃O-PEG-NH₂ 20000 (5.0 g, 0.25 mmole), previously azeotropically dried with 50 ml of toluene, and Et₃N (70 μl, 0.5 mmole). The solvent was then removed under vacuum and 40 ml of dry CH₂Cl₂ were added to the residue. The insoluble solid was removed by filtration and the filtrate was washed with pH 4.5 sodium chloride saturated acetate buffer. The organic phase was dried over anhydrous sodium sulfate, and the solution was concentrated till 15 ml under vacuum. The concentrated solution was dropped over 300 ml of diethyl ether under vigorously stirring. The precipitate was collected by filtration and dried under vacuum. Yield: 4.6 g (92%). ¹H-NMR (CDCl₃): δ 3.62 (bs, —O—CH₂—PEG), δ 3.54 (t, —CH₂—CH₂—NH—CO—NHS), δ 2.5 (t, —NH—CO—CH₂—CH₂—NH—CO—NHS), δ 2.8 (s, —NHS).

### Example 4

### Preparation of CH₃O—PEG-NH-(4-carboxymethyl)-piperidine-CO—NHS (CH₃O—PEG-NH—CMP—CO—NHS)

Reactions

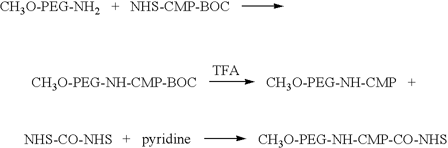

CH₃O—PEG-NH₂ 20000 (5.0 g, 0.25 mmole) was azeotropically dried with 50 ml of toluene and then slowly cooled to room temperature. To the resulting 12 ml solution were added 10 ml of dry CH₂Cl₂, N-hydroxysuccinimide ester of N—BOC-(4-carboxymethyl)-piperidine (CMP; 170 mg, 0.5 mmole) and Et₃N (70 μl, 0.5 mmole), and the reaction was let to proceed overnight at room temperature. The solution was then filtered and dropped over 300 ml of diethyl ether under vigorously stirring. The precipitate, collected by filtration and dried under vacuum, was then dissolved in 20 ml of a solution of CH₂Cl₂/TFA/H₂O (54.5:45.4:0.1) and stirred for 3 hour at room temperature. The solvent was removed under vacuum and to the obtained oil 20 ml of CH₂Cl₂ was added. To the resulting solution were added disuccinimidyl carbonate (265 mg, 1 mmole) and pyridine (0.25 ml), and the reaction was let to proceed overnight at room temperature. The solvent was then removed under vacuum and 40 ml of dry CH₂Cl₂ were added to the residue. The insoluble solid was removed by filtration and the filtrate was washed with pH 4.5 sodium chloride saturated acetate buffer. The organic phase was dried over anhydrous sodium sulfate, and the solution was concentrated till 15 ml under vacuum. The concentrated solution was dropped over 300 ml of diethyl ether under vigorously stirring. The precipitate was collected by filtration and dried under vacuum. Yield: 4.1 g (82%).

### Example 5

### Modification of Human Growth Hormone (hGH) with CH₃O—PEG-NH—CO—(CH₂)₂—NH—CO—NHS 5000

Reactions

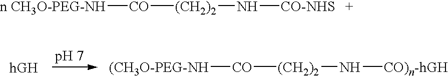

To 1 ml of a solution of hGH, 5 mg/ml in phosphate buffer 10 mM pH 7, 34.3 mg of CH₃O—PEG-NHOC—(CH₂)₂—NH—CO—NHS 5000 (6.85×10⁻³ mmole) were added. The solution was stirred and maintained at 5° C. for 2 hours. The reaction was stopped adding 5.14 mg (6.85×10⁻² mmole) of Gly. The solution was then filtered by 0.22 μm filter and analyzed directly.

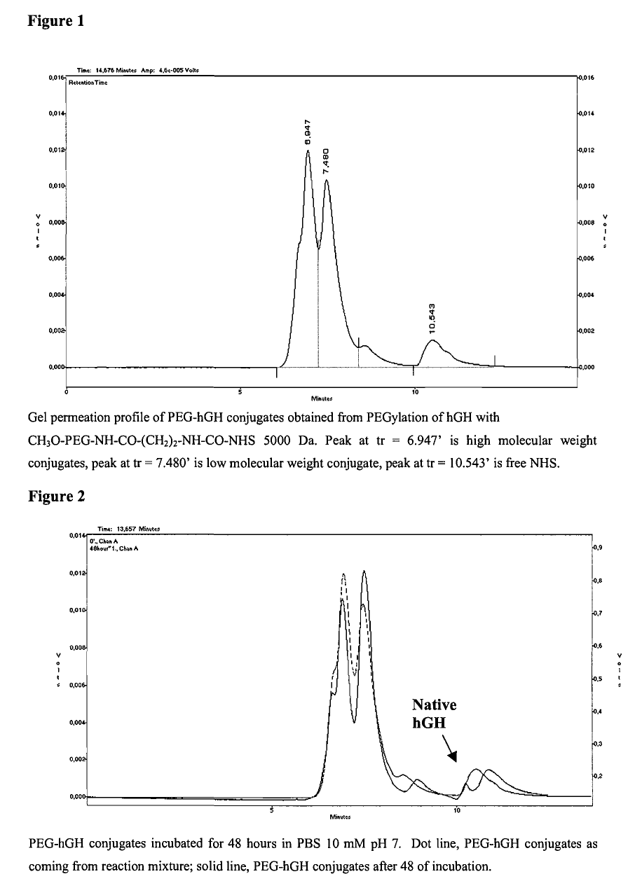

The solution obtained was investigated by gel permeation chromatography (GPC) and as shown in FIG. 1 by disappearance of native hGH peak (usually at tr=10.2′) all the protein amount reacted with PEG after 2 hours. Mainly, two conjugates were formed, one having a higher hydrodynamic volume (tr=6.947′) than the other (tr=7.480′), a difference due to different degree of PEGylation as confirmed by MALDI-TOF mass-spectrometry investigation. The peaks from gel permeation were collected and desalted and then analyzed by MALDI-TOF mass-spectrometry. From the analysis appeared that the peak at tr=7.480′ is mainly formed by monoPEG-hGH conjugates and the peak at tr=6.947′ is formed by diPEG-hGH and triPEG-hGH conjugates.

When the solution of the PEG-hGH conjugates, obtained as above reported, is incubated at room temperature the chromatogram profile in GPC shows within 48 hours a slow decrease of the peak area corresponding to di-, triPEG-hGH conjugates counterbalanced by an increase of the monoPEG-hGH peak and the formation of free hGH, peak at tr=10.2′ (FIG. 2).

The data suggest that only few chains (mainly 1 or 2) of the new PEG derivative react with hGH although the large equivalent excess of polymer (30 times) used in this conjugation. This is in contrast with that reported by Clark R. in a PEGylation study of human growth hormone with N-hydroxysuccinimide ester of poly(ethylene glycol) 5000 Da (mPEG-CO—NHS) using PEG/protein molar ratio ranging from 10 to 30 (Clark R. J Biol Chem 1996, 271(36), 21969-21977), where a wide mixture of isomers, mainly composed of multi-PEGylated conjugates (tetra-, penta- and esa-PEG-hGH), was obtained. This shows that the higher reactivity of mPEG-CO—NHS leads to multi PEGylation, because the polymer can also react with moderately reactive amino groups in the protein, thus making difficult both the discrimination among all modifiable amino groups and the obtainment of monoPEGylated species only. Furthermore, the conjugates synthesized by Clark don't release the attached PEG chains, this implies that the lost in protein activity after PEGylation is permanent, meanwhile the data of GPC of the conjugates, obtained with the PEG derivatives objects of this invention, after 5 days of incubation show a slow hydrolysis of PEG-hGH conjugates thus partially restoring the native fully active protein.

### Example 6

### Pharmacokinetic and Pharmacodynamic of PEG-hGH Conjugates Obtained as Reported in Example 5

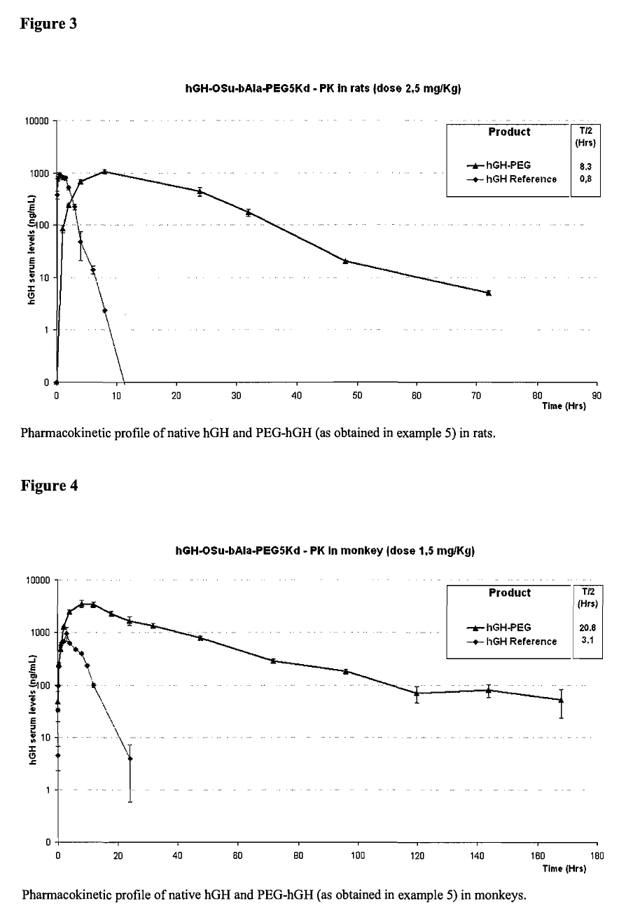

The pharmacokinetic profile of PEG-hGH conjugates, as obtained from example 5, was evaluated in rats and monkeys and compared to native hGH. The dose used was 2.5 mg/kg (expressed in protein) in the case of rats, and 1.5 mg/kg for the monkeys. The FIGS. 3 and 4 show the pharmacokinetic profiles of native hGH and PEG-hGH in rats and monkeys. The increment in term of half life (t_{1/2}), going from the native protein to the PEGylated form, was about 10 and 7 times in rats and monkeys, respectively. Data reported in literature (Clark R. J Biol Chem 1996, 271(36), 21969-21977) shown a t_{1/2}, of 5.8 hours for a diPEG₅₀₀₀-hGH and of 15 hours for a highly PEGylated pentaPEG₅₀₀₀-hGH, the conjugates analyzed in the study of Clark R. and coworkers were obtained by a multi-step purification of a wide mixture of multiPEGylated-hGH isomers. Both were obtained using mPEG₅₀₀₀-CO—NHS as activated PEG derivatives.

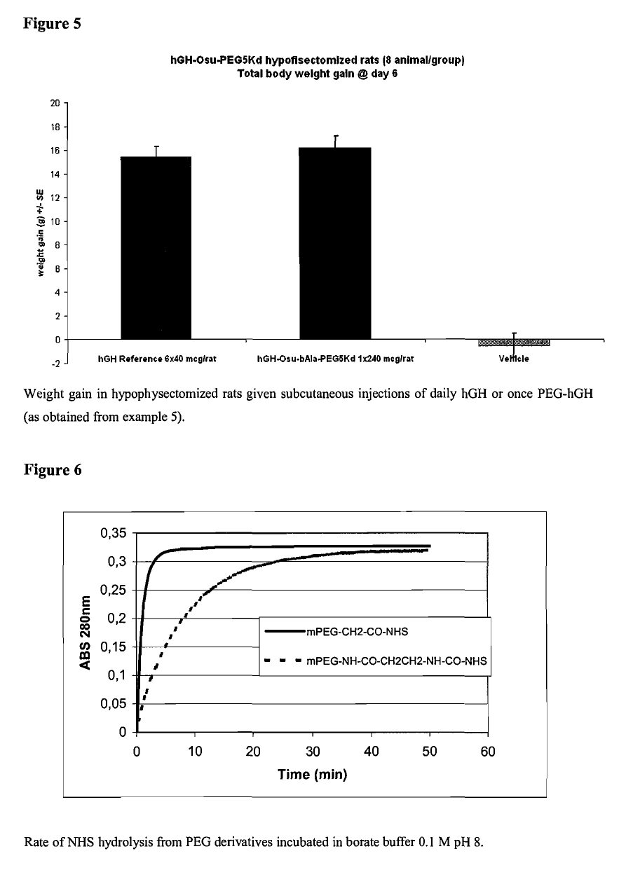

The pharmacodynamic was evaluated in hypophysectomized rats given subcutaneous daily injections of hGH, 6×40 μg/kg, or once injections of PEG-hGH (as obtained from example 5) 1 days×240 μg/kg. The animals' weight gain was followed for 6 days. As shown in FIG. 5 a single dose of PEG-hGH is equipotent as the daily injection of hGH.

### Example 7

### Stability and Reactivity of the New PEG Derivatives and mPEG-CH₂—CO—NHS

The stability and reactivity of the new PEG derivatives and mPEG-CH₂—CO—NHS was evaluated on the basis of N-hydroxysuccinimide (NHS) hydrolysis in water. The rate of NHS hydrolysis was followed by detecting the absorbance increase at 280 nm of a sample of activated PEG derivatives at 0.1 mmole in borate buffer 0.1 M pH 8. In FIG. 6 is shown the ABS 280 nm increase versus time for some PEG derivatives. It is clear the higher stability of mPEG-X—NH—CO—NHS derivatives with respect to the one of mPEG-CH₂—CO—NHS. In Table 1 is shown the NHS hydrolysis t_{1/2} for each PEG derivatives.

TABLE 1
Half life of NHS hydrolysis from different
NHS activated PEG derivatives.

| | NHS hydrolysis half |
|---|---|
| Conjugates | life (t½) (min) |
| mPEG-CH₂—CO—NHS | 1.05′ |
| mPEG-NH—CO—(CH₂)₂—NH—CO—NHS | 6.35′ |

The results of this experiment, which demonstrates the lower reactivity towards water of the new PEG derivatives, are in agreement with the lower degree of protein modification obtained using these derivatives (as reported in example 5) and, consequentially, only the most reactive and exposed residues in a protein can be modified.

### Example 8

### Comparison of Reactivity of Different PEG Derivatives Towards Single Amino Acid

To compare the reactivity of the CH₃O-PEG-NH—CO—(CH₂)₂—NH—CO—NHS and the reactivity of N-hydroxysuccinimide ester of mPEG (mPEG-CH₂—CO—NHS), the conjugation of these PEG polymers were studied towards N—BOC-Tyr, Nα-BOC-His and other protected amino acids possessing potentially reactive group such as the hydroxyl group.

A solution of the amino acid was prepared in CH₂Cl₂ at the final concentration of 7 mg/ml and the pH was brought to 8 with Et₃N. The PEG derivative was added in a molar ratio 1/5 with the respect to the amino acid equivalents. The degree of coupling was analyzed by RP-HPLC. The CH₃O—PEG-NH—CO—(CH₂)₂—NH—CO—NHS reacted only with Nα-BOC-His while, in the same condition, mPEG-CH₂—CO—NHS forms a conjugate with the hydroxy group, thus proving the lower reactivity of the new polymer that allows it to discriminate among different reactive groups in a protein. Furthermore, both derivatives form a conjugate with Nα-BOC-His (coupling to the N6, atom in the imidazole side chain) but the conjugate between mPEG-CH₂—CO—NHS and the amino acid is very unstable, in fact it reflects a typical activated polyethylene glycol the carbonyl imidazole PEG, meanwhile the conjugate obtained using CH₃O-PEG-NH—CO—(CH₂)₂—NH—CO—NHS is more stable and the degree of Nα-BOC-His release is about 35% over 5 days of incubation in water. This can be exploited in protein PEGylation using conditions that preferentially direct the PEG linking to His residue of a protein (as reported in Wang Y, et al. Adv Drug Del Rev 2002, 54, 547-570) thus obtaining conjugates sufficiently stable in vivo to achieve prolonged blood half-life but at the same time able to release partially the native protein or a conjugate of it with fewer attached PEG chains.

### Example 9

### Modification of LHRH Peptide with CH₃O—PEG-NH—CO—(CH₂)₂—NH—CO—NHS 5000

Reactions

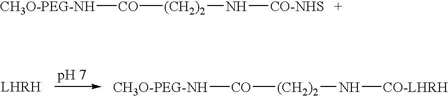

The LHRH peptide (P-GlyHisTrpSerTyrDTrpLeuArgProGly) is devoid of primary amino groups for PEG linking, and conjugation may take place only at the level of His side chain because the other potentially reactive amino acids (as the Tyr presents in the peptide) demonstrated, in separate experiment, no-reactivity towards the PEG derivatives objects of this invention.

To 1 ml of LHRH solution, 0.32 mg/ml in phosphate buffer 10 mM pH 7, 36.6 mg of CH₃O—PEG-NHOC—(CH₂)₂—NH—CO—NHS 5000 (7.32×10⁻³ mmole) were added. The solution was stirred and maintained at 5° C. for 2 hours. The reaction was stopped adding 5.49 mg (7.32×10⁻² mmole) of Gly. The solution was filtered by 0.22 μm filter and analyzed as follows:

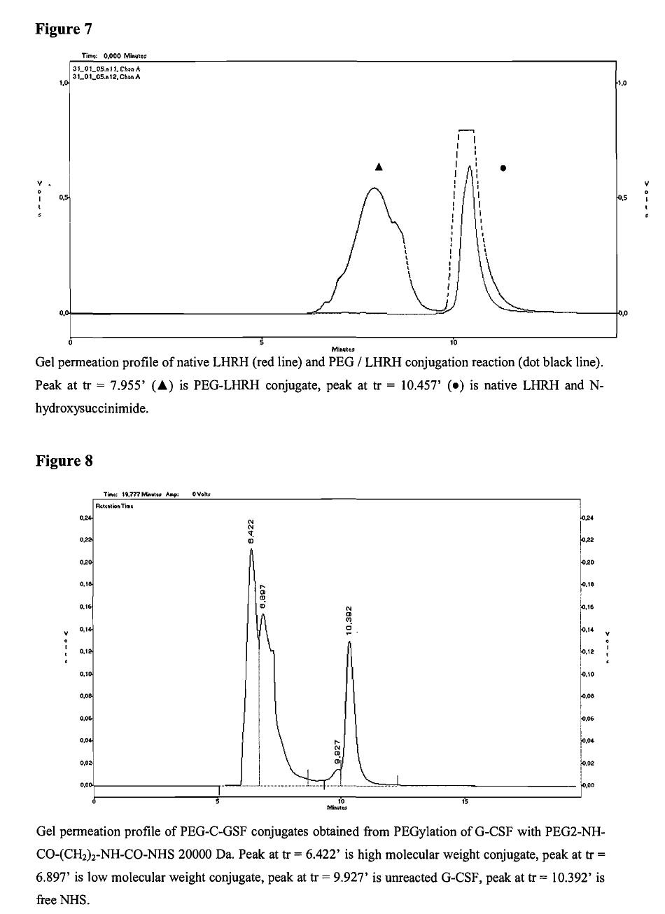

the conjugation was evaluated by GPC and, as shown in FIG. 7, the appearance of LHRH-PEG conjugate peak at 7.955° demonstrated a conjugate formation.

### Example 10

### Modification of Granulocyte Colony Stimulating Factor (G-CSF) with PEG2-NH—CO—(CH₂)₂—NH—CO—NHS 20000

Reactions

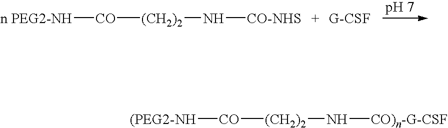

To 1 ml of a solution of G-CSF, 5 mg/ml in phosphate buffer 10 mM pH 7, 80.34 mg of PEG2-NH—CO—(CH₂)₂—NH—CO—NHS 20000 (4.02×10⁻³ mmole) were added. The solution was stirred and maintained at 5° C. for 2 hours. The reaction was stopped adding 3.01 mg (4.02×10⁻² mmole) of Gly. The solution was then filtered by 0.22 μm filter and the product obtained was directly analyzed by gel permeation chromatography as shown in FIG. 8. The analysis reveals that all G-CSF was PEGylated within 2 hours (disappearance on G-CSF peak at tr=9.927′) and at the same times two conjugates were formed, one having an higher hydrodynamic volume (tr=6.422′) than the other (tr=6.897′). The difference its due to different degree of polymer linking, evidently the first has more PEG chains linked to the protein than the second.

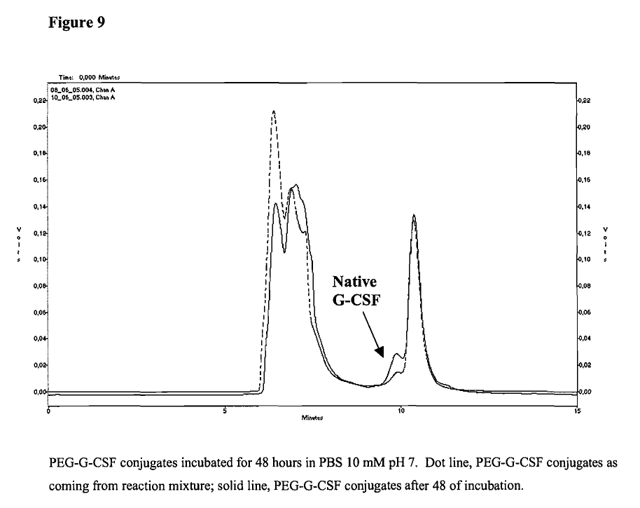

The solution of conjugates, as obtained above, was incubated at room temperature for 48 hours and following analyzed by GPC. The analysis showed a slow decrease of the peak area at tr=6.422′ (corresponding to high molecular weight conjugates) counterbalanced by an increase of the peak at tr=6.897′ (corresponding to low molecular weight conjugates) and the formation of free G-CSF, peak at tr=9.927′ (FIG. 9).

### Example 11

### Modification of Epirubicin with CH₃O—PEG-NH—CO—(CH₂)₂—NH—CO—NHS 5000

Reactions

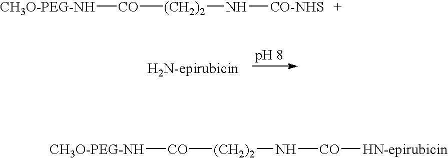

CH₃O—PEG-NH—CO—(CH₂)₂—NH—CO—NHS was conjugate to epirubicin to prepare a macromolecular prodrug that can be useful to prolong the body residence time of the small drug.

To 250 mg of epirubicin HCl (0.43 mmol), dissolved in 40 ml of DMF, 2.15 g of PEG-NH—CO—(CH₂)₂—NH—CO—NHS (0.358 mmol) were added. After PEG dissolution, 119.9 μl of Et₃N (0.86 mmol) were added to the reaction mixture. The reaction was let to proceed for 12 hours in dark and under stirring. About 30 ml of CH₂Cl₂ was then added and the unreacted epirubicin was extracted by HCl 0.1N (6×80 ml). The organic phase, dried over anhydrous Na₂SO₄, was concentrated to small volume. To the obtained oil, 15 ml of CH₂Cl₂ was added and the concentrated solution was dropped over 300 ml of diethyl ether, under vigorously stirring. The precipitate was collected by filtration and dried under vacuum. Yield: 1.82 g (0.328 mmol; 91.6%).

The invention has been described in particular exemplified embodiments. However, the foregoing description is not intended to limit the invention to the exemplified embodiments, and the skilled artisan should recognize that variations can be made within the scope and spirit of the invention as described in the foregoing specification.

On the contrary, the invention includes all alternatives, modifications, and equivalents that may be included within the true spirit and scope of the invention as defined by the appended claims.

1. A compound of formula Poly-(X—NH—CO-A)ₙ
wherein:
Poly is a hydrophilic polymer having a molecular weight of from about 300 to 100000 Daltons;
A together with —NH—CO— forms a reactive group;
X is a spacer moiety or a bond; and
n is an integer between 1 and 50.

2. The compound according to claim 1 wherein A is selected from the group consisting of: N-hydroxysuccinimide, N-hydroxybenzotriazole and p-nitrophenol.

3. The compound according to claim 1 wherein X is selected from the group consisting of:
a) —NH—CO—CH(R1)-CH(R2)
wherein R1 and R2, are each selected from the group consisting of: H, an optionally substituted alkyl group, an optionally substituted aryl group, an optionally substituted aryl-alkyl group, an hydroxy group, an amino group and a carboxy group;
b) an alkyl group optionally substituted with one or more hydroxy, amino, or carboxy groups; and
c) an aryl group.

4. The compound according to claim 1 wherein X is a C₂-C₁₀ alkyl group.

5. The compound according to claim 3 wherein R1, R2, or R1 and R2, are H.

6. The compound according to claim 3 wherein R1, R2, or R1 and R2, are a C₂-C₁₀ alkyl group.

7. The compound according to claim 1 wherein n is an integer between 1 and 10.

8. The compound according to claim 1 wherein n is 1.

9. The compound according to claim 1 wherein Poly is a linear or branched poly(ethylene glycol) or a derivative thereof.

10. The compound according to claim 9 wherein said derivative is selected from methoxy-poly(ethylene glycol) or diol-poly(ethylene glycol).

11. The compound according to claim 9 wherein said poly(ethylene glycol) has a molecular weight of from about 5000 to 60000 Daltons.

12. A method for manufacturing a conjugate between a pharmaceutically or diagnostically active agent and the compound according to claim 1.

13. The method according to claim 12 wherein said active agents are selected from peptides, oligonucleotides, proteins or non-peptide drugs.

14. A method for preparing a conjugate between a pharmaceutically or diagnostically active agent and the compound according to claim 1, said method comprising the steps of:
a) mixing the pharmaceutically or diagnostically active agent and the compound;
b) isolating the final conjugate.

15. The method according to claim 14, wherein said active agent is selected from the group consisting of: peptides, oligonucleotides, proteins and non-peptide drugs.

16. A method according to claim 14, wherein said active agent is selected from the group consisting of: hemoglobin, insulin, urokinase, alpha-interferon, G-CSF, hGH, asparaginase, adenosine deaminase, superoxide dismutase and catalase.

17. The method according to claim 14, wherein the mixing is carried out in water or buffered solutions.

18. The method according to claim 14, wherein the mixing is carried out at a temperature of between 3-40° C.

19. The method according to claim 14, wherein the mixing is carried out for 1-3 hours.

20. The method according to claim 14, wherein the isolation is performed by precipitation or by chromatographic techniques.

21. A conjugate prepared by the method of claim 14.

22. A pharmaceutical or a diagnostic composition comprising the conjugate of claim 21.

23. A composition according to claim 22 for oral, parenteral, rectal, topical, vaginal, ophthalmic or inhalation administration.

24. The composition according to claim 22 which is a water solution.

25. A compound according to claim 1 wherein n is an integer between 1 and 5.

26. A compound according to claim 9 wherein said poly(ethylene glycol) has a molecular weight of from about 10000 to 40000 Daltons.
